SEQUENCE LISTING (13 sequences)

SEQ ID NO: 1 - protein, 154 aa, Artificial Sequence
  Synthetic polynucleotide - Human eIF-5A1 amino acid sequence with K50R mutation
MADDLDFETGDAGASATFPMQCSALRKNGFVVLKGRPCKIVEMSTSKTGRHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 2 - DNA, 21 bp, Homo sapiens
gcuggacuccuccuacacatt

SEQ ID NO: 3 - protein, 154 aa, Homo sapiens
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 4 - DNA, 21 bp, Artificial Sequence
  Synthetic Oligonucleotide - siRNA
gcuggacuccuccuacacatt

SEQ ID NO: 5 - DNA, 21 bp, Artificial Sequence
  Synthetic Oligonucleotide - siRNA complement
uguguaggaggaguccagctt

SEQ ID NO: 6 - DNA, 31 bp, Artificial Sequence
  Primer
gaagcggccgcaccaccctgggccaggctgg

SEQ ID NO: 7 - DNA, 36 bp, Artificial Sequence
  Primer
ccacgcgtagaggtgttaaaaagtctttaggtaaag

SEQ ID NO: 8 - DNA, 28 bp, Artificial Sequence
  Primer
ccaactagtgcgaccgccaaaccttagc

SEQ ID NO: 9 - DNA, 31 bp, Artificial Sequence
  Primer
caaaagcttgacaacgtccgaggctccttgg

SEQ ID NO: 10 - DNA, 29 bp, Artificial Sequence
  Primer
gttatcgatactagtgcgaccgccaaacc

SEQ ID NO: 11 - DNA, 36 bp, Artificial Sequence
  Primer
caagcggccgccataccacatttgtagaggttttac

SEQ ID NO: 12 - DNA, 537 bp, Artificial Sequence
  Synthetic polynucleotide - Human eIF-5A1 nucleic acid sequence with K50R mutation
atagggagacccaagctggagaccatgtacccatacgacgtcccagactacgctggaagcttaatggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaggcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaataactggaattc

SEQ ID NO: 13 - DNA, 1355 bp, Homo sapiens
agtgcgggggtggagggcggaggagatagatagcacgcttgcgcggctgtcatagggctgcttggttggtcagtggggagtcggcgcctgcgtactaagacccgtgtgcagcagcggcggcggcggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccagggtggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtcttttttttttttttttttttaattcaatctggaatcagaaagcggtggattctggcaaatggtccttgtgccctccccactcatccctggtctggtcccctgttgcctatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaagcccccaa

(19) United States
(12) Patent Application Publication
(10) Pub. No.: US 2015/0126582 A1
(43) Pub. Date: May 7, 2015

(54) Combination Treatment of Multiple Myeloma

(71) Applicant: Sevion Therapeutics, Inc., La Jolla, CA (US)

(72) Inventors: Catherine A. Taylor, Waterloo (CA); Zhongda Liu, Waterloo (CA); Terence Tang, Waterloo (CA); Richard S. Dondero, Riverdale, NJ (US); John E. Thompson, Waterloo (CA)

(73) Assignee: Sevion Therapeutics, Inc., La Jolla, CA (US)

(21) Appl. No.: 14/398,112

(22) PCT Filed: Apr. 30, 2013
(86) PCT No.: PCT/US13/38803

§ 371 Date: Oct. 30, 2014

Related U.S. Application Data
(60) Provisional application No. 61/640,634, filed on Apr. 30, 2012.

(51) Int. Cl. A61K 31/713; A61K 38/17; A61K 47/48; A61K 31/454

(52) U.S. Cl. CPC A61K 31/713; A61K 31/454; A61K 38/1709; A61K 47/48192; A61K 48/00
USPC 514/44 A

(57) ABSTRACT

The present invention relates to the treatment of multiple myeloma in a human subject comprising administering lenadlidomide in combination with a vector which expresses a human eIF-SAI which is unable to be hypusinated and an siRNA which targets eIF-SAI. In some embodiments, the lenalidomide is administered simultaneously with the vector and the siRNA while in some embodiments the lenalidomide is administered at a time that is different from when the vector and the siRNA are administered. In some embodiments, the lenalidomide is administered orally and the vector and the siRNA are administered intraveneously.

### SEQUENCE LISTING SUBMISSION VIA EFS-WEB

A computer readable text file, entitled “061945-5035-WO-SequenceListing.txt,” created on or about Apr. 30, 2013 with a file size of about 8 kb contains the sequence listing for this application and is hereby incorporated by reference in its entirety.

### FIELD OF THE INVENTION

The present invention relates to the administration of a therapeutically effective amount of lenalidomide, siRNA targeting eIF-5A1, and a vector which expresses an eIF-5A protein which is unable to be hypusinated to induce apoptosis of malignant plasma cells in a subject suffering from multiple myeloma. The present invention also relates to pharmaceutical compositions comprising an effective amount of the combination of these agents.

### BACKGROUND OF THE INVENTION

Cancers, including multiple myeloma, are diseases which would benefit from the ability to induce apoptosis. Conventional therapies for multiple myeloma include chemotherapy, stem cell transplantation, high-dose chemotherapy with stem cell transplantation and salvage therapy. Chemotherapies include treatment with thalidomide, bortezomib, pamidronate, steroids and zoledronic acid. However many chemotherapy drugs are toxic to actively dividing non-cancerous cells, such as of the bone marrow, the lining of the stomach and intestines, and the hair follicles. Therefore, some types of chemotherapy may result in a decrease in blood cell counts, nausea, vomiting, diarrhea and loss of hair.

Conventional chemotherapy, or standard-dose chemotherapy, is typically the primary or initial treatment for patients with of multiple myeloma. Patients also may receive chemotherapy in preparation for high-dose chemotherapy and stem cell transplant. Induction therapy (conventional chemotherapy prior to a stem cell transplant) can be used to reduce the tumor burden prior to transplant. Examples of chemotherapy drugs suitable for induction therapy include lenalidomide (Revlimid®)/dexamethasone, thalidomide/dexamethasone, VAD (vincristine, Adriamycin® (doxorubicin), and dexamethasone in combination), and DVd (pegylated liposomal doxorubicin (Doxil® and Caelyx®), vincristine, and reduced schedule dexamethasone in combination).

The standard treatment for of multiple myeloma is melphalan in combination with prednisone (a corticosteroid drug), achieving a response rate of 50%. Unfortunately, melphalan is an alkylating agent and is less suitable for induction therapy. Corticosteroids (especially dexamethasone) are sometimes used alone for multiple myeloma therapy, especially in older patients and those who cannot tolerate chemotherapy. Dexamethasone is also used in induction therapy, alone or in combination with other agents. VAD is the most commonly used induction therapy, but DVd has recently been shown to be effective in induction therapy. However, none of the existing therapies offer a significant potential for a cure.

It has previously been demonstrated that when polynucleotides encoding eIF-5A are administered to malignant cancer cells, there is an increase in apoptosis those cells. It is therefore possible to induce cellular apoptosis by administering eIF-5A1 polynucleotides that are then expressed in malignant cells (see U.S. 20030050272). When cells accumulate the hypusinated form of eIF-5A1, the cells enter into a survival mode and do not undergo apoptosis as they normally would. Notably, in cancer cells, there is a significant amount of hyspusinated eIF-5A and thus, the cells do not enter into apoptosis. Thus, to treat cancer by killing the cancer cells (induce the cancer cells to enter into the apoptosis pathway), a polynucleotide encoding eIF-5A1 is administered to the subject or to the cancer cells or tumor to provide increased expression of eIF-5A1, which in turn causes apoptosis in the cancer cells and ultimately cell death.

The present invention encompasses this current gene therapy based approach in combination with chemotherapeutic approaches to produce a synergistic effect in killing malignant cancer cells. Previous disclosures did not contemplate this approach (see U.S. 20100004314).

### SUMMARY OF THE INVENTION

The invention encompasses a method of treating multiple myeloma in a human subject suffering therefrom comprising administering to the human subject a therapeutically effective amount of a combination of lenalidomide, a vector which expresses a human eIF-5A1 protein which is unable to be hypusinated, and an siRNA which targets eIF-5A1.

In some embodiments, the lenalidomide is administered simultaneously with the vector and the siRNA while in some embodiments the lenalidomide is administered at a time that is different from when the vector and the siRNA are administered. In some embodiments, the lenalidomide is administered orally and the vector and the siRNA are administered intraveneously.

In some embodiments, the vector encodes a human eIF-5A1 that contains at least one mutation selected from the group consisting of K50A, K50R, K67A, K47R, K67R, K50A/K67A, K50A/K47R, K50A/K67R, K50R/K67A, K50R/K47R, K50R/K67R, and K47A/K67A as set forth in SEQ ID NO: 3. In some embodiments, the vector encodes a human eIF-5A1 that contains a K50R mutation as set forth in SEQ ID NO: 1. In some embodiments, the vector is a pCpG plasmid. In some embodiments, the vector contains a polynucleotide encoding the eIF-5A1 which is linked to a B cell specific promoter. In some embodiments, the B cell specific promoter is B29.

In some embodiments, the siRNA targets SEQ ID NO: 2 and is double-stranded for 19-25 nucleotides in length. In some embodiments, the siRNA has at least one single-stranded overhang region, with each single-stranded region comprising six or fewer nucleotides. In some embodiments, the siRNA has two single-stranded overhang regions. In some embodiments, each of the two single-stranded overhang regions comprise two nucleotides or less. In some embodiments, one strand of the siRNA comprises the nucleotide sequence of 5′-GCUGGACUCCUCCUACACA-3′ (SEQ ID NO: 4). In some embodiments, one strand of the siRNA comprises the nucleotide sequence of 5′-UGUGUAGGAGGAGUCCAGC-3′ (SEQ ID NO: 5). In some embodiments, the vector and siRNA are independently complexed to polyethylenimine while in other embodiments, the vector and siRNA are together complexed to polyethylenimine.

The invention also encompasses a pharmaceutical composition comprising a therapeutically effective amount of lenalidomide, a vector which expresses a human eIF-5A1 protein which is unable to be hypusinated, and an siRNA which targets eIF-5A1. In some embodiments, the vector contains a pCpG plasmid which encodes a human eIF-5A1 that contains a K50R mutation as set forth in SEQ ID NO: 3 whose expression is linked to a B29 B cell specific promoter. In some embodiments, the siRNA comprises the nucleotide sequence of 5′-GCUGGACUCCUCCUACACA-3′ (SEQ ID NO: 4) and the complementary strand comprises the nucleotide sequence of 5′-UGUGUAGGAGGAGUCCAGC-3′ (SEQ ID NO: 5).

### DETAILED DESCRIPTION OF THE INVENTION

An embodiment of the invention encompasses methods of treating multiple myeloma which comprise administering to a subject in need of such treatment a therapeutically effective amount of 3-(4-amino-oxo-1,3-dihydro-isoindol-2-yl)-piperidine-2,6-dione (lenalidomide: see structure below), or a pharmaceutically acceptable salt, solvate, hydrate, stereoisomer, clathrate, or prodrug thereof, in combination with a therapeutically effective amount of the combination of a vector which expresses eIF-5A1 protein which cannot be hypusinated and an siRNA targeting eIF-5A1. This embodiment encompasses the treatment, prevention or management of multiple myeloma in the subject.

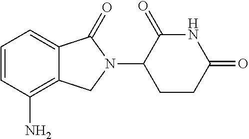 [SMILES: Nc1cccc2c1CN(C1CCC(=O)NC1=O)C2=O]

The structure and synthesis of lenalidomide are described in U.S. Pat. Nos. 5,635,517 and 7,465,800 which are hereby incorporated by reference. Lenalidomide has an asymmetric carbon atom and can exist as the optically active forms S(−) and R(+), and is produced as a racemic mixture with a net optical rotation of zero. In some embodiments the lenalidomide is administered as 25 mg once daily orally on Days 1-21 of repeated 28-day cycles. In some embodiments, the lenalidomide may be administered with a dose of dexamethasone of 40 mg once daily on Days 1 to 4, 9 to 12, and 17 to 20 of each 28-day cycle for the first 4 cycles of therapy and then 40 mg/day orally on Days 1 to 4 every 28 days. In some embodiments, the lenalidomide may be administered as capsules containing 2.5 mg, 5 mg, 10 mg, 15 mg or 25 mg of lenalidomide per capsule. Dosing schedules for reducing any toxic side effects when administering lenalidomide are described in U.S. Pat. No. 6,555,554 which is hereby incorporated by reference.

As used herein, SNS01-T is a complex of an expression vector comprising a polynucleotide encoding a mutant eIF-5A1 wherein the mutant eIF-5A1 is unable to be hypusinated, and an eIF-5A1 siRNA targeted against the 3′ end of eIF-5A1, wherein the expression vector and siRNA are complexed to polyethylenimine to form a complex. In certain embodiments, the polynucleotide encoding the mutant eIF-5A1 is eIF-5A1K50R (SEQ ID NO: 1) and the siRNA targets the sequence shown in SEQ ID NO: 2. The expression vector comprises a polynucleotide encoding a mutant eIF-5A1 and a promoter operably linked to provide expression of the polynucleotide in a subject. The promoter preferably is either tissue specific or systemic. For example, for treating multiple myeloma, it is preferable to use a B cell specific promoter, such as B29. In certain embodiments, the expression vector comprises a pCpG plasmid.

In certain embodiments, the expression vector comprising the mutant eIF-5A1 polynucleotide and the eIF-5A1 siRNA and are independently complexed to polyethylenimine, such as JetPEI™ (from commercially available sources). In other embodiments, the expression vector comprising the mutant eIF-5A1 polynucleotide and eIF-5A1 siRNA are complexed together to polyethylenimine. In some embodiments, the delivery vehicle comprises a polyethylenimine nanoparticle. An exemplary polyethylenimine nanoparticle is JetPEI™ currently produced by Polyplus Transfection. JetPEI™ is a cationic polymer transfection agent useful as a DNA and siRNA delivery agent. It contains a linear polyethylenimine reagent that provides reliable nucleic acid delivery in animals. The complexes and methods for producing the complexes are described in US 20100004314 which is hereby incorporated by reference.

JetPEI™ condenses nucleic acids into roughly 50 nm nanoparticles which are stable for several hours. As a result of this unique protection mechanism, aggregation of blood cells following injection is reduced compared to other reagents thereby preventing restricted diffusion within a tissue, erythrocyte aggregation and microembolia. These nanoparticles are sufficiently small to diffuse into the tissues and enter the cells by endocytosis. JetPEI™ favors nucleic acids release from the endosome and transfer across of the nuclear membrane.

In some embodiments, the vector is a plasmid, and both the plasmid and siRNA are administered to the subject via a JetPEI™ complex. The plasmid comprising the polynucleotide and siRNA maybe complexed together via a polymer complex such as polyethylenimine or the JetPEI™ complex or may separately complexed to a polymer. For example, where the plasmid comprising the polynucleotide and siRNA are to be administered separately to the subject (separately in the meaning of time and/or delivery site) it is preferable to have the plasmid and siRNA complexed to a different carrier. Where the administration will be at the same time and at the same site, it may be preferable to complex the plasmid and siRNA together.

As used herein, multiple myeloma, also known as plasma cell myeloma or Kahler's disease, refers to a cancer of plasma cells, a white blood cell normally responsible for producing antibodies. In multiple myeloma, collections of abnormal plasma cells accumulate in the bone marrow, where they interfere with the production of normal blood cells. Most cases of myeloma also feature the production of a paraprotein, an abnormal antibody which can cause renal failure. Bone lesions and hypercalcemia are also often encountered in subjects suffering from multiple myeloma. The invention encompasses a method of treatment for multiple myeloma in a subject suffering from multiple myeloma which may include alleviation of any of these symptoms associated with this disease.

In some embodiments, treatment of multiple myeloma includes, but is not limited to, inducing apoptosis in malignant plasma cells, thereby killing these cells, and reducing the number of malignant plasma cells. The method comprises administering a composition comprising eIF-5A1 siRNA and a polynucleotide encoding a mutant eIF-5A1. The composition and eIF-5A1 siRNA and a polynucleotide encoding a mutant eIF-5A1 are discussed herein below.

All cells produce eIF-5A (also referred to as factor 5A). Mammalian cells produce two isoforms of eIF-5A (eIF-5A1 and eIF-5A2). eIF-5A1 has been referred to as apoptosis-specific eIF-5A, as is upregulated in cells undergoing apoptosis. Human eIF-5A1 has the accession number NP 001961 (SEQ ID NO: 3) for amino acid sequence and NM 001970 (SEQ ID NO: 13) for nucleic acid sequence. eIF-5A1 is responsible for shuttling out of the nucleus subsets of mRNA encoding proteins necessary for apoptosis. eIF-5A2 has been referred to as proliferation eIF-5A as it is responsible for shuttling out of the nucleus subsets of mRNA encoding proteins necessary for cellular proliferation (see Rosorius (1999) J. Cell Science, 112, 2369-2380).

Both eIF-5A1 and eIF-5A2 are post translationally modified by deoxyhypusine synthase (DHS). DHS hypusinates eIF-5A. Hypusine, a unique amino acid, is found in all examined eukaryotes and archaebacteria, but not in eubacteria, and eIF-5A is the only known hypusine-containing protein (Park (1988) J. Biol. Chem., 263, 7447-7449). Hypusinated eIF-5A is formed in two post-translational steps: the first step is the formation of a deoxyhypusine residue by the transfer of the 4-aminobutyl moiety of spermidine to the alpha-amino group of a specific lysine of the precursor eIF-5A catalyzed by deoxyhypusine synthase. The second step involves the hydroxylation of this 4-aminobutyl moiety by deoxyhypusine hydroxylase to form hypusine.

In some embodiments of the invention, the polynucleotide encoding a mutated eIF-5A1 can be mutated so that it cannot be hypusinated and thus will not be available to stimulate the cell into survival mode. For example, in one embodiment, the polynucleotide encoding eIF-5A is mutated so that the lysine at position 50 (K50), which is normally hypusinated by DHS, is changed to an alanine (K50A) or arginine (K50R) which cannot be hypusinated.

In another embodiment, the lysine at position 67 is changed to an arginine (K67R). In another embodiment the lysine at position 67 is changed to an alanine (K67A). In another embodiment, the lysine at position 47 is changed to an arginine (K47R).

In other embodiments, a double mutant is used. One double mutant is where the lysine at position 50 is changed to an arginine (K50R) and the lysine at position 67 is changed to an arginine (K67R). This double mutant is referred to as K50R/K67R.

Another double mutant is where the lysine at position 47 is changed to an arginine (K47R) and the lysine at position 50 is changed to an arginine (K50R). This mutant is referred to as K47R/K50R. The invention provides another double mutant where the lysine at position 50 is changes to an alanine (K50A) and the lysine at position 67 is changes to an alanine (K67A). This mutant is referred to as K50A/K67A. The invention provides another double mutant where the lysine at position 50 is changes to an arginine (K50R) and the lysine at position 67 is changes to also changed arginine (K67R). This mutant is referred to as K50R/K67R.

In some embodiments, local delivery of siRNA may be utilized. If the siRNA is delivered locally to the malignant plasma cell, then the expression is preferably knocked out. By knocking out expression, there is no eIF-5A around that can be hypusinated and thus there is no hypusinated eIF-5A to lock the cells into survival mode. Since the siRNA is delivered locally to the site of the malignant plasma cell (e.g. bone marrow), there is no need to have eIF-5A available for regular cell growth.

In certain embodiments, the siRNA targeted against eIF-5A1 is administered to the subject to suppress expression of the endogenous eIF-5A1. In certain embodiments the siRNA comprises SEQ ID NO: 4 or SEQ ID NO: 5 or is any siRNA targeted against eIF-5A1 that will suppress expression of endogenous eIF-5A1. In certain embodiments, the eIF-5A1 is human eIF-5A1 (shown in SEQ ID NO: 3) and the subject is a human. Other siRNA targeted against human eIF-5A1 are known and disclosed in US 20110098460 which is hereby incorporated by reference. In certain embodiments, the siRNA consists of the siRNA construct shown in SEQ ID NO: 4 or SEQ ID NO: 5. For example, the siRNA contains nucleic acids targeted against the eIF-5A1 but also contains overhangs such as U or T nucleic acids or also contains tags, such as a histidine tag. Molecules or additional nucleic acids attached at either the 5′ or 3′ end may be included and fall within the “consisting of” as long as the siRNA construct is able to reduce expression of the target gene. Preferably the siRNA targets regions of the eIF-5A1 gene so as to not effect expression of the exogenous polynucleotide. For example the eIF-5A1 siRNA targets the 3′ UTR or the 3′ end of the polynucleotide encoding the eIF-5A1 gene.

As used herein, the term “treatment” indicates a procedure which is designed ameliorate one or more causes, symptoms, or untoward effects of multiple myeloma in a subject. Likewise, the term “treat” is used to indicate performing a treatment. The treatment can, but need not, cure the subject, i.e., remove the cause(s), or remove entirely the symptom(s) and/or untoward effect(s) of multiple myeloma. Thus, a treatment may include treating a subject to inhibit the growth or proliferation of malignant plasma cells in the subject, or it may attenuate symptoms such as, but not limited to, blood hypercalcemia, renal failure caused by high levels of monoclonal proteins (M proteins) in the blood or Bence Jones proteins in the urine, anemia-related fatigue, and osteolytic bone damage and fractures. These four problems are often referred to by the acronym CRAB, which refers to calcium levels, renal failure, anemia and bone damage.

As used herein, the term “subject” is used interchangeably with the term “patient” and is used to mean an animal, in particular a mammal, and even more particularly a human.

Methods of treating multiple myeloma described herein comprise administering a pharmaceutically effective amount of lenalidomide in combination with SNS01-T to a subject suffering from multiple myeloma. As used herein, the term “administer” and “administering” are used to mean introducing lenalidomide and SNS01-T into a subject. When administration is for the purpose of treatment, the substance is provided at, or after the onset of, a symptom of multiple myeloma. The therapeutic administration of this substance serves to attenuate any symptom, or prevent additional symptoms from arising. When administration is for the purposes of preventing or reducing the progression of multiple myeloma, the substance is provided in advance of any visible symptom, but after a diagnosis of multiple myeloma by the detection of M proteins in the blood or Bence Jones proteins in the urine. The prophylactic administration of the substance serves to attenuate subsequently arising symptoms or prevent or reduce the likelihood of the symptoms from arising altogether.

The route of administration of the lenalidomide includes, but is not limited to, oral (such as a capsule), subcutaneous, intra-arterial, intravenous, intramuscular, intra-osseous, intra-peritoneal and intrathecal. In an exemplary embodiment, the route of administration is oral. The route of administration of SNS01-T includes, but is not limited to, intravenous, subcutaneous, intra-arterial, intramuscular, intra-osseous, intra-peritoneal and intrathecal. In an exemplary embodiment, the route of administration is intraveneous. In some embodiments, the lenalidomide is administered via a route of administration distinct from the vector and siRNA may also be administered the same route as the vector and siRNA. For example, lenalidomide may be administered orally while SNS01-T is administered intravenously.

The methods of treating multiple myeloma of the present invention also relate to co-administering lenalidomide and SNS01-T to the subject. The term “co-administer” indicates that each of at least two compounds are administered during a time frame wherein the respective periods of biological activity or effects overlap. Thus, the term includes sequential as well as coextensive administration of compounds. Similar to administering compounds, co-administration of more than one substance can be for therapeutic and/or prophylactic purposes. If more than one substance or compound is co-administered, the routes of administration of the two or more substances need not be the same.

As used herein and unless otherwise indicated, the phrase “therapeutically effective amount” (or “pharmaceutically effective amount”) of lenalidomide and SNS01-T or a pharmaceutically acceptable salt thereof is measured by the therapeutic effectiveness, wherein at least one adverse effect of a disorder is ameliorated or alleviated. In one embodiment, the term “therapeutically effective amount” means an amount of lenalidomide and SNS01-T that is sufficient to provide the desired local or systemic effect and performance at a reasonable benefit/risk ratio attending any medical treatment (e.g. chemotherapeutic toxicity). The response to the therapeutically effective amount may be a cellular, organ or tissue-specific response, or system or systemic response. In one embodiment, the phrase “therapeutically effective amount” is measured by the therapeutic effectiveness of lenalidomide and SNS01-T to alleviate at least one symptom associated with multiple myeloma. In some embodiments, the effects of the combination of SNS01-T are not merely additive but synergistic. Examples of therapeutically effective amounts include, but are not limited to those in the Examples section herein.

As used herein and unless otherwise indicated, “pharmaceutically acceptable” refers to materials and compositions that are physiologically tolerable and do not typically produce an allergic or similar untoward reaction, such as gastric upset, dizziness and the like, when administered to a human. Typically, as used herein, the term “pharmaceutically acceptable” means approved by a regulatory agency of the Federal or a state government or listed in the U.S. Pharmacopeia or other generally recognized pharmacopeia for use in animals, and more particularly in humans.

The phrase “pharmaceutically acceptable salts” as used herein includes but is not limited to salts of acidic or basic groups that may be present in lenalidomide. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds, included in the present compositions, which are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

Pharmaceutical formulations of the invention can take the form of solutions, suspensions, emulsion, tablets, pills, pellets, capsules, capsules containing liquids, powders, sustained-release formulations, suppositories, emulsions, suspensions, or any other form suitable for use. In one embodiment, the pharmaceutically acceptable vehicle for lenalidomide is a capsule (see U.S. Pat. No. 7,119,106) while the pharmaceutically acceptable vehicle for SNS01-T is an intraveneous solution. Other examples of suitable pharmaceutical vehicles are described in Remington's The Science and Practice of Pharmacy (2010). The pharmaceutical compositions may contain preserving agents, solubilising agents, stabilising agents, wetting agents, emulsifiers, sweeteners, colorants, odorants, salts, buffers, coating agents or antioxidants, such as but not limited to butylated hydroxytoluene (BHT). They may also contain therapeutically active agents in addition to the substance of the present invention.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example 1: Production of SNS01-T

Preparation of 1 mL SNS01-T in microfuge tube

1. Add 21.8 μL of 2.3 mg/mL pExp5A to a sterile eppendorf tube.

2. Add 25 μL of siRNA to the eppendorf tube containing the pDNA. Use the pipette tip to gently mix by pipetting up and down slowly 5 times.

3. Add 203 μL of 11.3 mM Tris-HCl pH 7.4 to the DNA/siRNA mixture. Use the pipette tip to gently mix by pipetting up and down slowly 5 times.

4. Add 250 μL of 10% glucose. Use the pipette tip to gently mix by pipetting up and down slowly 10-12 times.

5. Set tube aside and proceed to the next step.

6. Add 9 μL of invivo-jetPEI to separate sterile eppendorf tube.

7. Add 241 μL of 11.3 mM Tris-HCl pH 7.4 to the eppendorf tube containing the invivo-jetPEI. Use the pipette tip to gently mix by pipetting up and down slowly 5 times.

8. Add 250 μL of 10% glucose to the invivo-jetPEI/tris mixture. Use the pipette tip to gently mix by pipetting up and down slowly 10-12 times.

9. Using a P1000 set to 1000 μL volume, transfer the entire volume from the tube containing the DNA/siRNA/tris/glucose mixture from Step A to the tube containing the PEI/tris/glucose mixture from Step B. Use the pipette tip to gently mix by pipetting up and down slowly 10-12 times. Set tube aside at room temperature and allow a minimum of 30 minutes for nanoparticle formation prior to use.

CpG-free Cloning Vectors and pCpG Plasmids were obtained from commercially available sources. These plamids are completely devoid of CpG dinucleotides, as thus designated pCpG. These plasmids yield high levels of transgene expression both in vitro and in vivo, and in contrast to CMV-based plasmids allow sustained expression in vivo. pCpG plasmids contain elements that either naturally lack CpG dinucleotides, were modified to remove all CpGs, or entirely synthesized such as genes encoding selectable markers or reporters. Synthesis of these new alleles was made possible by the fact that among the sixteen dinucleotides that form the genetic code, CG is the only dinucleotide that is non-essential and can be replaced. Eight codons contain a CG encoding for five different amino acids. All eight codons can be substituted by at least a choice of two codons that code for the same amino acid to create new alleles that code for proteins having amino acid sequences that remain identical to the wild type and thus are as active as their wild-type counterparts. These new alleles are available individually in a plasmid named pMOD from which they can be easily excised.

The empty vector, pCpG-mcs (InvivoGen) is a vector with no expressed gene product, only a multiple cloning site, and was used as the control vector. The pExp5A expression vector was used to express eIF5K50R under the control of a B cell specific (B29) promoter and enhancer. To create pExp5A, the pCpG-mcs plasmid was digested with EcoRl to remove the mammalian expression cassette and then ligated to a synthetic linker with a new multiple cloning site. The B29 DHS4.4 3′ enhancer was amplified from KAS-6/1 genomic DNA using the following primers: 5′-GAAGCGGCCGCACCACCCTGGGCCAGGCTGG-3′ (SEQ ID NO: 6) and 5′-CCACGCGTAGAGGTGTTAAAAAGTCTTTAGGTAAAG-3′ (SEQ ID NO: 7), and ligated into the NotI and MluI sites of the linker (pCpG-DHS4.4). The −167 B29 promoter was amplified from pDrive-hB29 (Invivogen) using primers: 5′ CCAACTAGTGCGACCGCCAAACCTTAGC-3′ (SEQ ID NO: 8) and 5′-CAAAAGCTTGACAACGTCCGAGGCTCCTTGG-3′ (SEQ ID NO: 9), and ligated into the SpeI and HindIII sites of pCpG-LacZ. The resulting plasmid was used to amplify the B29-eIF5AK50R expression cassette using the primers: 5′-GTTATCGATACTAGTGCGACCGCCAAACC-3′ (SEQ ID NO: 10) and 5′-CAAGCGGCCGCCATACCACATTTGTAGAGGTTTTAC-3′ (SEQ ID NO: 11), and ligated into the ClaI and NotI sites in the multiple cloning site of pCpG-DHS4.4 to create pExp5A.

The DNA was prepared under GLP conditions by VGXI. Endotoxin levels measured and are <10 EU/mg; DNA was at 2.3 mg/ml in sterile water.

The control siRNA used in the experiments was a micro-array validated non-targeting control siRNA from Dharmacon (D-001810-01).

The eIF-5A1 siRNA used in the experiments was designed against the 3′UTR of human eIF-5A1. There is no similarity between the human eIF-5A1 siRNA and mouse eIF5A1 and the siRNA should therefore only suppress human (but not mouse) eIF-5A1. The siRNA also has no similarity to eIF-5A2 (either human or mouse). The eIF5A1 siRNA targets the following sequence: 5′-GCUGGACUCCUCCUACACA(TT)-3′ (SEQ ID NO: 2) in the 3′-UTR. The siRNA was formulated at 1 mg/mL in water.

Tris-HCl was produced at 11.3 mM at a pH of 7.4 under GLP conditions by KBI.

Glucose was produced at a concentration of 10% under GLP conditions by KBI.

### Example 2: Co-Administration of Lenalidomide with eIF-5A1 Plasmid and eIF-5A1 siRNA (SNS01-T) in SCID Mice with Multiple Myeloma Subcutaneous Tumors

SCID mice are injected subcutaneously with RPMI 8226 cells. Treatment is initiated when palpable tumors are observed. Groups of six SCID/CB17 mice (3-5 weeks old) are injected with 12 million RPMI 8226 myeloma cells in 200 μL PBS in their right flank and treatment is initiated when the tumours reached a minimum size of 10 mm³.

Control mice are injected intravenously two times per week with PEI complexes containing pCpG-mcs (empty vector) and control siRNA at a dose of 0.375 mg (nucleic acid)/kg. Mice receiving SNS01-T were injected intra-venously two times per week at 0.375 mg/kg. Mice treated with lenalidomide received 5 doses per week by intra-peritoneal injection of lenalidomide (Celgene) at doses of 15 mg/kg or 50 mg/kg. Mice receiving both SNS01-T and lenalidomide were dosed 2×/week with SNS01-T by intra-venous injection at 0.375 mg/kg and 5× per week with lenalidomide by intra-peritoneal injection at either 15 mg/kg or 50 mg/kg. Mice that did not receive SNS01-T received an intra-venous injection of 5% glucose/5 mM Tris-HCl vehicle. Mice that did not receive lenalidomide received an intra-peritoneal injection of DMSO/PBS vehicle. Tumor volume is measured for all the mice in each group at least twice weekly.

At the end of 6 weeks of dosing, tumor growth was inhibited compared to control nanoparticles by 96% (p=0.0001), 94% (p=0.0002), and 99% (p=0.00003) in animals treated with SNS01-T, SNS01-T plus 15 mg/kg lenalidomide and SNS01-T plus 50 mg/kg of lenalidomide, respectively. By comparison, tumor inhibition in mice treated with 5 doses/week of lenalidomide for 6 weeks at 15 mg/kg i.p. or 50 mg/kg i.p. was 51% (p=0.03) and 78% (p=0.0008), respectively.

No surviving animals treated with control nanoparticles or lenalidomide alone had undetectable tumors at the end of 6 weeks. In the SNS01-T and SNS01-T plus 15 mg/kg of lenalidomide groups, 2 of 5 animals in each group had no detectable tumor. In the SNS01-T plus 50 mg/kg treatment group, 5 of 6 animals (83%) had no detectable tumor, and remained undetectable even after 3 weeks without further treatment.

The median survival of mice treated with control nanoparticles or 15 mg/kg lenalidomide was 48 days and 53 days, respectively. Mice treated with SNS01-T or SNS01-T in combination with lenalidomide had 100% survival following 6 weeks of dosing and 11 days of observation after cessation of treatment.

SNS01-T alone and in combination with sub-optimal and optimal (15 and 50 mg/kg respectively) doses of lenalidomide demonstrated significantly improved efficacy compared to lenalidomide alone. However, the most significant finding was that SNS01-T plus 50 mg/kg of lenalidomide completely eliminated tumor burden in 83% of treated animals compared to 40% in animals treated with SNS01-T alone or SNS01-T plus 15 mg/kg lenalidomide. The eradication of tumor has lasted for at least 3 weeks.

In an additional study, mice implanted with human myeloma tumors derived from RPMI 8226 cells were randomized into 4 groups and treated with control nanoparticles, or, either SNS01-T (0.375 mg/kg; i.v., twice per week), lenalidomide (50 mg/kg; i.p., 5× per week) or both. The mice received 2 cycles of treatment, 6 weeks and 5 weeks respectively, with an 11 day rest period between cycles. Mice, whose tumors were undetectable at the end of cycle 1, received no drug treatment in cycle 2 unless tumor reappeared. The mice were monitored for 2 weeks after the end of the second cycle of treatment. The total length of the study was 102 days.

At the end of the second cycle of dosing, tumor growth was inhibited compared to control nanoparticles by 84% (p<0.0001), 34% (p=0.05), and 98.1% (p<<0.0001) in animals treated with SNS01-T, 50 mg/kg of lenalidomide, and SNS01-T plus 50 mg/kg of lenalidomide, respectively. The median survival of mice treated with control nanoparticles or 50 mg/kg of lenalidomide was 48 days and 95 days, respectively. Mice treated with SNS01-T or SNS01-T in combination with lenalidomide had 60% and 100% survival, respectively, and thus median survival could not be determined in these groups.

In conclusion, SNS01-T alone and in combination with 50 mg/kg of lenalidomide demonstrated significantly improved efficacy compared to lenalidomide alone. Tumors were eradicated in 4 out of 6 animals receiving SNS01-T plus lenalidomide and did not reappear during 8 weeks with no further treatment.

Although the present invention has been described in detail with reference to examples above, it is understood that various modifications can be made without departing from the spirit of the invention. Accordingly, the invention is limited only by the following claims.

1. A method of treating multiple myeloma in a human subject suffering therefrom comprising administering to the human subject a therapeutically effective amount of a combination of lenalidomide, a vector which expresses a human eIF-5A1 protein which is unable to be hypusinated, and an siRNA which targets eIF-5A1.

2. The method of claim 1, wherein the lenalidomide is administered simultaneously with the vector and the siRNA.

3. The method of claim 1, wherein the lenalidomide is administered at a time that is different from when the vector and the siRNA are administered.

4. The method of claim 1 wherein the lenalidomide is administered orally and the vector and the siRNA are administered intraveneously.

5. The method of claim 1 wherein the vector encodes a human eIF-5A1 that contains at least one mutation selected from the group consisting of K50A, K50R, K67A, K47R, K67R, K50A/K67A, K50A/K47R, K50A/K67R, K50R/K67A, K50R/K47R, K50R/K67R, and K47A/K67A as set forth in SEQ ID NO: 3.

6. The method of claim 1 wherein the vector encodes a human eIF-5A1 that contains a K50R mutation as set forth in SEQ ID NO: 1.

7. The method of claim 1 wherein the vector is a pCpG plasmid.

8. The method of claim 1 wherein the vector contains a polynucleotide encoding the eIF-5A1 which is linked to a B cell specific promoter.

9. The method of claim 8 wherein the B cell specific promoter is B29.

10. The method of claim 1 wherein the siRNA targets SEQ ID NO: 2 and is double-stranded for 19-25 nucleotides in length.

11. The method of claim 1 wherein the siRNA has at least one single-stranded overhang region, with each single-stranded region comprising six or fewer nucleotides.

12. The method of claim 1 wherein the siRNA has two single-stranded overhang regions.

13. The method of claim 12 wherein each of the two single-stranded overhang regions comprise two nucleotides or less.

14. The method of claim 1 wherein one strand of the siRNA comprises the nucleotide sequence of 5′-GCUGGACUCCUCCUACACA-3′ (SEQ ID NO: 4).

15. The method of claim 1 wherein one strand of the siRNA comprises the nucleotide sequence of 5′-UGUGUAGGAGGAGUCCAGC-3′ (SEQ ID NO: 5).

16. The method of claim 1 wherein the vector and siRNA are independently complexed to polyethylenimine.

17. The method of claim 1 wherein the vector and siRNA are together complexed to polyethylenimine.

18. A pharmaceutical composition comprising a therapeutically effective amount of lenalidomide, a vector which expresses a human eIF-5A1 protein which is unable to be hypusinated, and an siRNA which targets eIF-5A1.

19. The pharmaceutical composition of claim 18 wherein the vector contains a pCpG plasmid which encodes a human eIF-5A1 that contains a K50R mutation as set forth in SEQ ID NO: 3 whose expression is linked to a B29 B cell specific promoter.

20. The pharmaceutical composition of claim 18 wherein one strand of the siRNA comprises the nucleotide sequence of 5′-GCUGGACUCCUCCUACACA-3′ (SEQ ID NO: 4) and the complementary strand comprises the nucleotide sequence of 5′-UGUGUAGGAGGAGUCCAGC-3′ (SEQ ID NO: 5).
